(19) United States
(12) Patent Application Publication
(10) Pub. No.: US 2003/0176475 A1
(43) Pub. Date: Sep. 18, 2003

(54) Therapeutically useful new salts of cck inhibitors, process for the preparation thereof and pharmaceutical preparations containing them

(76) Inventors: Istvan Hermecz, Budapest (HU); Agnes Horvath, Budapest (HU); Gyulane Kiss, Budapest (HU); Miklos Morvai, Mogyorod (HU); Benjamin Podanyi, Dunakeszi (HU); Kalman Simon, Budapest (HU); Judit Sipos, Budapest (HU); Agota Smelkone Esek, Budapest (HU); Anna Szabo, Budapest (HU); Arpadne Vasvari, Budapest (HU)

Correspondence Address: SANOFI-SYNTHELABO INC., 9 GREAT VALLEY PARKWAY, P.O. BOX 3026, MALVERN, PA, 19355, US

(21) Appl. No.: 10/240,698

(22) PCT Filed: Apr. 4, 2001
(86) PCT No.: PCT/HU01/00039

(30) Foreign Application Priority Data
Apr. 7, 2000 (HU) P 0001417

(51) Int. Cl. A61K 31/427; C07D 417/02

(52) U.S. Cl. 514/370; 548/181

(57) ABSTRACT

New salts of the general formula (I), their solvates, polymorphs and pseudo polymorphs wherein X stands for ethanolamine, diethanolamine or diethylamine.

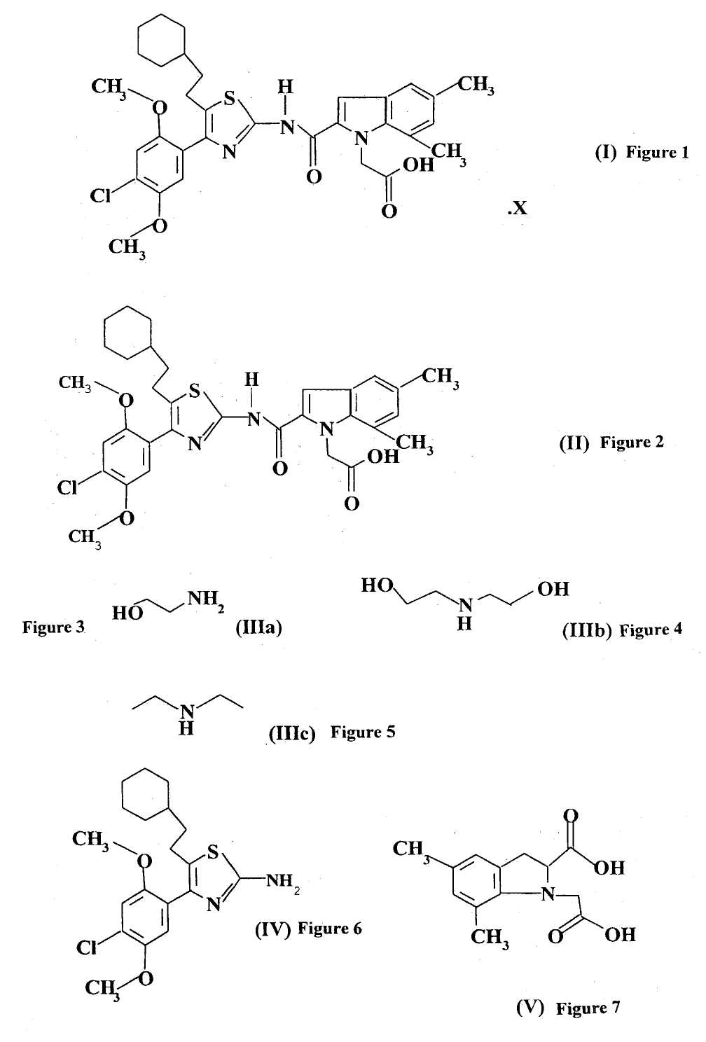

The invention relates to the therapeutically useful new salts of the general formula (I) (FIG. 1), their solvates, polymorphs and pseudopolymorphs, to the process for the preparation thereof and to the pharmaceutical preparations containing them, wherein in the formula

X means ethanolamine, diethanolamine or diethylamine.

The compound of the formula (II) (FIG. 2) is a cholecystokinin A (CCK-A) agonist, which can be used for the treatment of the disorders of the gastrointestinal system, and of the central nervous system.

The following salts of the compound of the formula (II) and the processes for their preparation are described in publication WO 99/15525: salt with trifluoroacetic acid, salt with hydrochloric acid, mono- and di-sodium salts, mono- and di-potassium salts. The salt with trifluoroacetic acid and its hydrate, because of the strong toxicity of the trifluoroacetic acid, are not suitable for therapeutic use. The composition of the salt with hydrochloric acid is not stochiometric, it is not reproducible.

The mono- and di-sodium salts, as well as the mono- and di-potassium salts and their hydrates are highly hygroscopic and chemically not stable compounds namely the compound of the formula (II) decomposes at the amide bond giving rise to decomposition products of formulae (IV) (FIG. 6) and (V) (FIG. 7).

The subjects of the present invention, the therapeutically applicable new salts of the general formula (I) and their solvates, polymorphs and pseudopolymorphs wherein in the formula X has the meaning as given above, with special regards to the ethanolamine salt and its solvates, are chemically stable, non-hygroscopic and their preparation is well reproducible.

Preparation of the subjects of the present invention, the therapeutically applicable new salts of the general formula (I) and their solvates, polymorphs and pseudopolymorphs is carried out by reacting the 2-[[[4-(4-chloro-2,5-dimethoxyphenyl)-5-(2-cyclohexylethyl)-2-thiazolyl]amino]carbonyl]-5,7-dimethyl-1H-indol-1-acetic acid of the formula (II) with the ethanolamine of the formula (IIIa) (FIG. 3) or the diethanolamine of the formula (IIIb) (FIG. 4) or the diethylamine of the formula (IIIc) (FIG. 5).

The compounds (IIIa), (IIIb) or (IIIc) are preferably applied in an excess of 1,2-1,5 equivalents. The reaction is preferably carried out in protic solvents, at a temperature between 40-100° C., preferably at the boiling point of the solvent. As for protic solvents preferably ethanol, acetonitrile or ethanol-water mixture can be applied.

The therapeutically applicable new salts of the general formula (I) and their solvates, polymorphs and pseudopolymorphs of the present invention are cholecystokinin A (CCK-A) agonists, which can be used for the, treatment of the disorders of the gastrointestinal system, and of the central nervous system.

The agonistic effect of the compound according to the present invention was investigated as described in the publication WO/99/15525 by investigating its effect on emptying the stomac, on rat, p.o. We have found that the ED₅₀ value, 34 μg/kg p.o, is in the same order as the ED₅₀ value of the trifluoroacetic acid salt and of the potassium salt.

A further subject of the present invention are pharmaceutical preparations containing the salts of the general formula (I), their solvates, polymorphs and pseudopolymorphs, as active ingredients.

The pharmaceutical, preparations according to the present invention, beside the active ingredients, may contain the usual pharmaceutical excipients and may be formulated as oral, sublingual, subcutan, intramuscular, intravenous, topical, intratracheal, intra-vasal, transdermal, rectal, intraocular, etc drug products.

The daily dose may be 0.01-50 mg bodyweight kg/day depending on the severity of the illness, on the sex and weight of the patient.

Further details of the invention are demonstrated by the following examples without limiting the invention to the examples.

### EXAMPLES

1. Preparation of 2-[[[4-(4-chloro-2,5-dimethoxyphenyl)-5-(2-cyclohexylethyl)-2-thiazolyl]amino]carbonyl]-5,7-dimethyl-1H-indol-1-acetic Acid Ethanolamine Salt

647.4 g (1.060 mol) of 2-[[[4-(4-chloro-2,5-dimethoxyphenyl)-5-(2-cyclohexylethyl)-2-thiazolyl]amino]carbonyl]-5,7-dimethyl-1H-indol-1-acetic acid and 7000 cm³ of ethanol are mixed and the resulting suspension is heated to 72° C. Then 95.3 cm³ (97.2 g=1.592 mol) of 2-aminoethanol are added to the suspension. The reaction mixture is heated to reflux temperature and refluxed for 30 minutes. The resulting pale yellow suspension is allowed to cool to room temperature, under stirring. The crystals are filtered off, washed with ethanol, dried in vacuum drying cupboard at 60-65° C.

Product: 694.6 g white crystals of the title compound, mp: 208-210° C.

2. Preparation of 2-[[[4-(4-chloro-2,5-dimethoxyphenyl)-5-(2-cyclohexylethyl)-2-thiazolyl]amino]carbonyl]-5,7-dimethyl-1H-indol-1-acetic Acid Diethanolamine Salt

1.22 g (2 mmol) of 2-[[[4-(4-chloro-2,5-dimethoxyphenyl)-5-(2-cyclohexylethyl)-2-thiazolyl]amino]carbonyl]-5,7-dimethyl-1H-indol-1-acetic acid and 18 cm³ of ethanol are mixed and the resulting suspension is heated to 70-75° C. and 0.4 cm³ (4 mmol) of diethanolamine are added to the suspension. The mixture is refluxed for 20 minutes. The resulting pale yellow suspension is allowed to cool to room temperature, under stirring. After cooling the crystals are filtered off, washed with ethanol, then with di-isopropyl ether.

Product: 1.29 gwhite crystals of the title compound, mp: 215.5-216.5° C.

3. Preparation of 2-[[[4-(4-chloro-2,5-dimethoxyphenyl)-5-(2-cyclohexylethyl)-2-thiazolyl]amino]carbonyl]-5,7-dimethyl-1H-indol-1-acetic Acid Diethylamine Salt

1.22 g (2 mmol) of 2-[[[4-(4-chloro-2,5-dimethoxyphenyl)-5-(2-cyclohexylethyl)-2-thiazolyl]amino]carbonyl]-5,7-dimethyl-1H-indol-1-acetic acid and 12 cm³ of acetonitrile are mixed, the resulting suspension is heated to 50° C. and 0.62 cm³ (4 mmol) of diethylamine are added to it. The mixture is stirred at 50° C. for 20 minutes. The pale yellow suspension is allowed to cool to room temperature, under stirring. After cooling the crystals are filtered off, washed with acetonitrile, then with di-isopropyl ether.

Product: 1.28 g white crystals of the title compound, mp: 209.4-210.7° C.

4. Preparation of 2-[[[4-(4-chloro-2,5-dimethoxyphenyl)-5-(2-cyclohexylethyl)-2-thiazolyl]amino]carbonyl]-5,7-dimethyl-1H-indol-1-acetic Acid Ethanolamine Monohydrate Salt

5 g (8.2 mmol) of 2-[[[4-(4-chloro-2,5-dimethoxyphenyl)-5-(2-cyclohexylethyl)-2-thiazolyl]amino]carbonyl]-5,7-dimethyl-1H-indol-1-acetic acid are added to the mixture of 45 cm³ of 96% ethanol and 30 cm³ of water. The resulting suspension is heated to 82° C., then 0.74 cm³ (13.2 mmol) of ethanolamine are added to it. The mixture is refluxed for 15 minutes. The pale yellow suspension is allowed to cool to room temperature, under stirring. The crystals are filtered off, washed with 96% ethanol.

Product: 5 g white crystals of the title compound, mp: 166-167/198-201° C.

What we claim is:

1) New salts of the general formula (I), their solvates, polymorphs and pseudo polymorphs—wherein X stands for ethanolamine, diethanolamine or diethylamine—.

2) 2-[[[4-(4-chloro-2,5-dimethoxy-phenyl)-5-(2-cyclohexyl-ethyl)-2-thiazolyl]amino]carbonyl]-5,7-diethyl-1H-indol-1-acetic acid ethanolamine salt, its solvates, polymorphs and pseudopolymorphs.

3) Process for the preparation of the new salts of the general formula (I), their solvates, polymorphs and pseudopolymorphs—wherein X stands for ethanolamine, diethanolamine or diethylamine—,characterized by reacting 2[[[4-(4-chloro-2,5-dimethoxy-phenyl)-5-(2-cyclohexyl-ethyl)-2 thiazolyl]amino]carbonyl]-5,7-diethyl-1H-indol-1-acetic acid of the formula (II) with ethanolamine of the formula (IIIa), or diethanolamine of the formula (IIIb) or diethylamine of the formula (IIIc).

4) Process according to claim 3 characterized by using the compounds of the formulaes (IIIa), (IIIb) or (IIIc) in an excess of 1,2-1,5.

5) Process according to claims 3 and 4 characterized by carrying out the reaction in a protic solvent.

6) Process according to claim 5 characterized by using as protic solvent ethanol, acetonitrile or an ethanol-water mixture.

7) Process according to claims 3 to 6 characterized by carrying out the reaction at the boiling point of the solvent.

8) Pharmaceutical composition containing as active ingredient a new salt of the general formula (I), its solvates, polymorphs or pseudopolymorphs—wherein X stands for ethanolamine, diethanolamine or diethylamine—.

9) Pharmaceutical composition according to claim 8 containing as active ingredient 2-[[[4-(4-chloro-2,5-dimethoxy-phenyl)-5-(2-cyclohexyl-ethyl)-2-thiazolyl]amino]carbonyl]-5,7-diethyl-1H-indol-1-acetic acid ethanolamine salt, its solvates, polymorphs or pseudopolymorphs.

10) Use of the pharmaceutically acceptable new salts of the general formula (I), its solvates, polymorphs or pseudopolymorphs—wherein X stands for ethanolamine, diethanolamine or diethylamine—.for the preparation of pharmaceutical compositions suitable for the treatment of the disorders of the gastrointestinal or central nervous systems.

11) Method of treatment of the disorders of the gastrointestinal or central nervous systems using an effective amount of a new salt of the general formula (I), its solvates, polymorphs or pseudopolymorphs—wherein X stands for ethanolamine, diethanolamine or diethylamine—.
